(19) United States
(12) Patent Application Publication
(10) Pub. No.: US 2026/0069650 A1
(43) Pub. Date: Mar. 12, 2026

(54) COMPLEX POLYSACCHARIDE FOR REGULATING TH1/TH2 IMMUNE BALANCE AND PREPARATION METHOD AND USE THEREOF

(71) Applicant: Infinitus (China) Company Ltd., Jiangmen (CN)

(72) Inventors: Ying ZHU, Jiangmen (CN); Jian TANG, Jiangmen (CN)

(21) Appl. No.: 18/939,112

(22) Filed: Nov. 6, 2024

(30) Foreign Application Priority Data
Sep. 9, 2024 (CN) 2024112628316

(51) Int. Cl. A61K 36/8969; A61K 36/03; A61K 36/04; A61K 36/07; A61K 36/355; A61K 36/484; A61P 37/08

(52) U.S. Cl. CPC A61K 36/8969; A61K 36/03; A61K 36/04; A61K 36/071; A61K 36/355; A61K 36/484; A61P 37/08; A61K 2236/33; A61K 2236/331; A61K 2236/53

(57) ABSTRACT

The present disclosure relates to a complex polysaccharide for regulating Th1/Th2 immune balance and a preparation method and use thereof. The complex polysaccharide is prepared from medicinal and edible homologous plants and edible fungi as raw materials. The raw materials include Agaricus Blazei Murill, seaweed, glycyrrhiza, polygonatum and honeysuckle; and compared with current drugs for treating ovalbumin-induced immune stress, the complex polysaccharide can be taken for a long time without obvious toxic or side effects. Moreover, the complex polysaccharide obtains a molecular weight segment effective to immunomodulation through co-extraction, and has obvious synergistic benefits compared to the immunomodulatory effect of the single polysaccharide. The complex polysaccharide can regulate the Th1/Th2 immune balance, prevent and/or inhibit the ovalbumin-induced immune stress response, providing effective support for the wide application of the ovalbumin.

### CROSS-REFERENCE TO RELATED APPLICATION

This application claims the benefit of priority to Chinese Patent Application No. 202411262831.6, filed Sep. 9, 2024, the entire disclosure of which is incorporated herein by reference.

### TECHNICAL FIELD

The present disclosure belongs to the technical field of biological medicines, and specifically relates to a complex polysaccharide for regulating Th1/Th2 immune balance and a preparation method and use thereof.

### BACKGROUND

There are two immune cell balance states in animals, namely, Th1/Th2 immune cell balance, which complement each other for the immune process in vivo, but also inhibit each other, keeping cellular immunity and humoral immunity in a balanced state. Generally. Th1 mediates intracellular immune responses to directly attack pathogens or promote other cells to attack the pathogens. Even if the pathogens enter infected cells, they will still launch an attack. Th2 mainly mediates humoral immune responses. Th2 cells will not enter infected cells, and they will promote other immune cells to produce antibodies, such as IL-13 and IL31, which are one of their signature antibodies, and these antibodies attack exogenous substances. Th1/Th2 imbalance will lead to different immune diseases in the body, such as rheumatoid arthritis and multiple sclerosis, and however when being dominant, Th2 cells will lead to allergic diseases, asthma, dermatitis, etc.

The main proteins in egg white include ovalbumin, ovotransferrin, ovomucoid, ovomucin, lysozyme, globulin G2, globulin G3, etc. The ovalbumin is the main protein component in the egg white, which is a phosphate glycoprotein, accounting for 54%-69% of the total proteins in the egg white, and is the most important protein in the egg white. The ovalbumin consists of 386 amino acid residues, has a relative molecular mass of approximately 45,000, and an isoelectric point of 4.5.

The ovalbumin is multiple-purpose. For example, it is an ingredient that can enhance human immunity, and the humoral immunity and cellular immunity of the human body are both composed of proteins, so the immune function can be improved by supplementing the ovalbumin. At the same time, the ovalbumin can not only increase the water retention capacity of the skin and improve the moisturizing effect, but also adjust the oil on the surface of the skin to an appropriate amount, so that the skin tissues more tightly integrated; with rich nutritional values and good functional properties, the ovalbumin can be used in food processing. For example, it has a strong emulsifying effect and can be used as an emulsifier to be applied in food processing: in addition, the ovalbumin also has good interfacial adsorption ability and can form a gel to be applied in the food processing industry. The ovalbumin is also widely used in baby food, meat processing industry, bread and dessert manufacturing industries due to its safe source, excellent foamability, hydrophilicity, emulsibility and easy gel formation. Recently, the ovalbumin has also been widely used in research on drugs and vaccine preparations, such as ovalbumin liposome transdermal vaccines, nanoparticle antibacterial agents, nano-lipid carriers, microsphere pulse sustained release systems, and nanoparticle nasal delivery systems.

Although the ovalbumin has a very wide range of applications, it is one of the main allergens in the egg white. Currently, in order to reduce its immune stress response, the ovalbumin is mostly modified by glycosylation and enzymatic hydrolysis to obtain hypoallergenic ovalbumin. However, some people are still prone to immune stress reactions, manifested as allergies, after taking foods and preparations containing the ovalbumin and its modified proteins. In view of the allergic phenomenon, currently chemical drugs are mostly adopted for antagonism, including loratadine, cetirizine hydrochloride, calamine lotion, hydrocortisone cream and the like. However, they all have certain side effects, such as thirst, headache, occasionally abnormal liver function, and even systemic anaphylaxis.

In recent years, multiple studies have shown that plant polysaccharides (also including edible fungus polysaccharides) have a good regulatory effect on the body's immunity, and thus are gradually used in clinical treatments or to improve the body's immune status. However, the efficacy of this single plant polysaccharide is often very limited under general application conditions.

In this regard, the patent CN101518556A discloses a polysaccharide composition for enhancing humoral immunity and cellular immune response in an ovalbumin-immunized mouse, which can induce immune responses and enhance mouse immunity. In addition, the patent CN109463742A discloses a polysaccharide formula capable of enhancing human immunity and a preparation process thereof, which can also achieve the effect of enhancing human immunity. However, at the moment, how to deal with the immune stress state caused by ovalbumin as an immunity-enhancing adjuvant and how to deal with the induced allergic phenomena are still lacking in the field of polysaccharide immune regulation.

### SUMMARY

An objective of the present disclosure is to provide a complex polysaccharide capable of regulating Th1/Th2 immune balance, and preventing and/or inhibiting an ovalbumin-induced immune stress response.

The technical solutions adopted by the present disclosure are as follows.

A first aspect of the present disclosure provides a polysaccharide composition including a polysaccharide extract of an active pharmaceutical ingredient, wherein the active pharmaceutical ingredient includes Agaricus Blazei Murill, seaweed, glycyrrhiza, polygonatum and honeysuckle.

In some embodiments of the present disclosure, by mass percentage, the active pharmaceutical ingredient includes: 20%-40% of Agaricus Blazei Murill, 10%-30% of seaweed, 5%-25% of glycyrrhiza, 5%-25% of polygonatum and 5%-20% of honeysuckle.

In some embodiments of the present disclosure, by mass percentage, the active pharmaceutical ingredient includes: 30%-40% of Agaricus Blazei Murill, 10%-20% of seaweed, 10%-25% of glycyrrhiza, 15%-25% of polygonatum and 5%-15% of honeysuckle.

In some embodiments of the present disclosure, by mass percentage, the active pharmaceutical ingredient includes: 20%-40% of Agaricus Blazei Murill, 10%-16% of seaweed, 22%-25% of glycyrrhiza, 20%-25% of polygonatum and 5%-20% of honeysuckle.

In some embodiments of the present disclosure, by mass percentage, the active pharmaceutical ingredient includes: 20%-30% of Agaricus Blazei Murill, 10%-16% of seaweed, 22%-25% of glycyrrhiza, 20%-22% of polygonatum and 5%-10% of honeysuckle.

In some embodiments of the present disclosure, the seaweed is selected from at least one of Phaeophyta, Rhodophyta, Laminaria japonica, and Undaria pinnatifida Suringar.

In some embodiments of the present disclosure, the glycyrrhiza is selected from at least one of Glycyrrhiza uralensis Fisch., Glycyrrhiza inflata Batal., and Glycyrrhiza glabra L.

In some embodiments of the present disclosure, the polysaccharide composition has a molecular weight of 0.5-2432 KDa.

In some embodiments of the present disclosure, the monosaccharide composition of the polysaccharide composition includes mannose, rhamnose, galactose, glucose, xylose, arabinose, fucose, and galacturonic acid.

In some embodiments of the present disclosure, the monosaccharide composition of the polysaccharide composition includes 0.5%-1.5% of mannose, 1%-2% of rhamnose, 0.2%-0.6% of galactose, 75%-85% of glucose, 12%-14% of xylose, 0.5%-1.2% of arabinose, 1.5%-2.5% of fucose, and 0.2%-0.6% of galacturonic acid.

In some embodiments of the present disclosure, the structure of the polysaccharide composition is a mixed crystalline and amorphous lamellar structure containing tertiary helices.

Agaricus Blazei Murill, also known as Agaricus subrufescens, is a medicinal and edible fungus, belongs to Basidiomycotina, Hymenomycetes, Agaricales, Agaricaceae, Agaricus, and is rich in protein and polysaccharide. Researches have shown that Agaricus Blazei Murill polysaccharide has antiviral, antioxidant and immune-modulating effects.

Seaweed contains a large amount of seaweed polysaccharide, which is a polymer compound composed of multiple identical or different monosaccharide groups linked by glycosidic bonds. Phaeophyta polysaccharides, fucoidan, Rhodophyta polysaccharides and the like all belong to this category. The seaweed polysaccharide has good gelling properties, stability, and film-forming properties, and also has various biological activities such as antiviral, anticoagulant, antitumor, immune regulation, and antioxidant.

Glycyrrhiza is a Chinese herbal medicine with a long history and widely used in medicine, food and industrial production. The glycyrrhiza contains polysaccharides, flavonoids and saponins, where the glycyrrhiza polysaccharide is an active polysaccharide extracted therefrom, easily soluble in water, shows therapeutic activity against inflammation and other diseases caused by infection, and also has anti-tumor, anti-viral, antioxidant, anti-inflammatory and other effects.

Polygonatum is the dried rhizome of the Liliaceae plant Polygonatumkingianum, P. sibiricum, or P. cyrtomema. Modern researches have shown that the polygonatum polysaccharide is one of the main active ingredients in the polygonatum and has anti-tumor, antioxidant, anti-inflammatory and antibacterial effects, regulating blood sugar and blood lipids, anti-virus, enhancing immunity and improving memory.

Honeysuckle is the dried flower bud or newly opened flower of Lonicera japonica Thumb., which is listed as top grade in Sheng Nong's herbal classic. It is cold in nature, sweet in taste, and has the effects of clearing heat and removing toxicity, and dissipating wind-heat. The honeysuckle polysaccharide extracted therefrom has anti-tumor, antioxidant, anti-viral, immunomodulatory, hypoglycemic and hypolipidemic effects.

It has found in the present disclosure that mixing the Agaricus Blazei Murill, seaweed. glycyrrhiza, polygonatum and honeysuckle at the above proportion to prepare the complex polysaccharide can regulate the Th1/Th2 balance, and prevent and/or inhibit ovalbumin-induced immune stress reactions.

A second aspect of the present disclosure provides a preparation method of a polysaccharide composition according to the first aspect of the present disclosure, which includes the following steps: mixing raw materials after being crushed to obtain a mixture, adding water to soak the mixture for extraction, collecting a filtrate for concentration, alcohol precipitation, and collecting a precipitate.

In some embodiments of the present disclosure, after being crushed, the raw materials pass through 20 meshes to 100 meshes.

In some embodiments of the present disclosure, a material-to-liquid ratio of the raw materials to water is 1:10-20 g/mL.

In some embodiments of the present disclosure, the soaking time is 20 min to 40 min.

In some embodiments of the present disclosure, the extraction is performed for 2 h to 4 h at 70° C. to 90° C.

In some embodiments of the present disclosure, filtration is also included after extraction; and specifically, a 200 meshes to 400 meshes of filter cloth is used for filtration.

In some embodiments of the present disclosure, the alcohol precipitation is that 3-5 volume times of absolute ethanol is added to stand still for 10 h to 14 h.

A third aspect of the present disclosure provides use of the polysaccharide composition described in the first aspect of the present disclosure in any of the following:
(1) preparation of products for regulating the Th1/Th2 balance;
(2) preparation of products for the prevention and/or treatment of Th1/Th2 imbalance-related diseases; and
(3) preparation of products for enhancing immunity.

In some embodiments of the present disclosure, the Th1/Th2 imbalance-related diseases include allergies and/or allergy-related diseases.

In some embodiments of the present disclosure, the allergy-related diseases include allergic rhinitis, allergic asthma, and the like.

In some embodiments of the present disclosure, the body allergy includes but is not limited to body ovalbumin-induced allergy.

In some embodiments of the present disclosure, the product further includes excipients acceptable in food, medicines, and health care products.

A fourth aspect of the present disclosure provides a product comprising the polysaccharide composition according to the first aspect of the present disclosure.

In some embodiments of the present disclosure, the product includes at least one of food, medicines and health care products.

In some embodiments of the present disclosure, the function of the product is to regulate Th1/Th2 balance or prevent and/or treat Th1/Th2 imbalance-related diseases and/or improve immunity.

Through direct preparation or preparation after adding pharmaceutically acceptable excipients, the above-mentioned polysaccharide composition can be prepared into any pharmaceutically acceptable dosage form. Taking an oral preparation as an example, the dosage form includes: a capsule, a tablet, a powder, a granule, a tea or an oral liquid. The drugs in various dosage forms above can be prepared according to conventional methods in the pharmaceutical field.

A fifth aspect of the present disclosure provides a method including administering the composition in the first aspect of the present disclosure or the product in the third aspect of the present disclosure to a subject; and the method is used for any of the following:
(1) regulating the Th1/Th2 balance;
(2) preventing and/or treating Th1/Th2 imbalance-related diseases; or
(3) improving immunity.

In some embodiments of the present disclosure, the Th1/Th2 imbalance-related diseases include allergies or allergy-related diseases.

In some embodiments of the present disclosure, the allergy-related diseases include allergic rhinitis, allergic asthma, and the like.

In some embodiments of the present disclosure, the allergy includes, but is not limited to, body ovalbumin-induced allergy.

In some embodiments of the present disclosure, a suitable dosage range of the composition per day is 1-500 mg/kg body weight; and the above dosage may be administered as a single dosage unit or in several dosage units, depending on the physician's clinical experience and the dosage regimen including the use of other therapeutic modalities.

In some embodiments of the present disclosure, when administered to animals, the daily administration dosage of the composition is 100-300 mg/kg/d, and/or, the administration time is 2-4 weeks, and/or, the administration methods include but are not limited to gavage, direct oral administration, and injection.

A “subject” is defined herein to include animals, such as mammals, including but being not limited to primates (e.g., humans), cattle, sheep, goats, horses, dogs, cats, rabbits, rats, mice, and the like.

The beneficial effects of the present disclosure are as follows.

The present disclosure provides a complex polysaccharide prepared from medicinal and edible homologous plants and edible fungi as raw materials. The raw materials include Agaricus Blazei Murill, seaweed, glycyrrhiza, polygonatum and honeysuckle; and compared with current drugs for treating ovalbumin-induced immune stress, the complex polysaccharide in the present disclosure can be taken for a long time without obvious toxic or side effects. Moreover, the complex polysaccharide in the present disclosure obtains a molecular weight segment effective to immunomodulation through co-extraction, and has obvious synergistic benefits compared to the immunomodulatory effect of the single polysaccharide. The complex polysaccharide in the present disclosure can regulate the Th1/Th2 immune balance, prevent and/or inhibit the ovalbumin-induced immune stress response, and provides effective support for the wide application of the ovalbumin.

### BRIEF DESCRIPTION OF DRAWINGS

FIG. 1 shows molecular weight determination of a complex polysaccharide in Example 1.

FIG. 2 is shows SEM (Scanning Electron Microscope) scanning of a complex polysaccharide in Example 1.

FIG. 3 shows polysaccharide Congo red tests in Comparative examples 1-5 and Example 1.

FIG. 4 shows an expression level of mouse serum IL-13 and IL-31; compared with a model group, *P<0.05, and compared with an Example 1 group, #p<0.05.

FIG. 5 shows expression levels of mouse serum IgE and HIS; compared with a model group, * P<0.05; and compared with an Example 1 group. #p<0.05.

FIG. 6 shows a comparison of rhinitis conditions in Example 1 and Comparative examples 1-5.

### DETAILED DESCRIPTION

The concepts and the generate technical effects of the present disclosure are clearly and completely described below in combination with embodiments, so as to fully understand the purposes, features and effects of the present disclosure. It is apparent that the described embodiments are only a part of the embodiments of the present disclosure but not all. Based on the embodiments of the present disclosure, all the other embodiments obtained by those of ordinary skill in the art on the premise of not contributing creative effort should belong to the protection scope of the present disclosure.

### Example 1

A complex polysaccharide was prepared by mixing the following raw materials in mass percentages: 40% of Agaricus Blazei Murill, 10% of Rhodophyta, 25% of Glycyrrhiza glabra L., 20% of polygonatum and 5% of honeysuckle.

The complex polysaccharide was prepared by the following process: (1) the above raw materials were taken and crushed separately to pass through a 45-mesh sieve for later use; (2) the raw material powder was taken according to the percentage of the composition components and mixed to obtain a mixture, and the mixture was added in deionized water with a material-to-liquid ratio of 1:15 (g/mL), and soaked for 30 min; (3) the material in step (2) was extracted at 80° C. for 3 h, filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a complex polysaccharide.

### Example 2

A complex polysaccharide was prepared by mixing the following raw materials in mass percentages: 20% of Agaricus Blazei Murill, 10% of Phaeophyta, 25% of Glycyrrhiza uralensis Fisch., 25% of polygonatum and 20% of honeysuckle.

The complex polysaccharide was prepared by the following process: (1) the above raw materials were taken and crushed separately to pass through a 45-mesh sieve for later use; (2) the raw material powder was taken according to the percentage of the composition components and mixed to obtain a mixture, and the mixture was added in deionized water with a material-to-liquid ratio of 1:20 (g/mL), and soaked for 30 min; (3) the material in step (2) was extracted at 90° C. for 3 h, filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a complex polysaccharide.

### Example 3

A complex polysaccharide was prepared by mixing the following raw materials in mass percentages: 30% of Agaricus Blazei Murill, 16% of Undaria pinnatifida Suringar, 22% of Glycyrrhiza inflata Batal., 22% of polygonatum and 10% of honeysuckle.

The complex polysaccharide was prepared by the following process: (1) the above raw materials were taken and crushed separately to pass through a 45-mesh sieve for later use; (2) the raw material powder was taken according to the percentage of the composition components and mixed to obtain a mixture, and the mixture was added in deionized water with a material-to-liquid ratio of 1:10 (g/mL). and soaked for 30 min; (3) the material in step (2) was extracted at 70° C. for 3 h, filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a complex polysaccharide.

### Example 4

A complex polysaccharide was prepared by mixing the following raw materials in mass percentages: 40% of Agaricus Blazei Murill, 30% of Undaria pinnatifida Suringar, 10% of Glycyrrhiza glabra L., 5% of polygonatum and 15% of honeysuckle.

The complex polysaccharide was prepared by the following process: (1) the above raw materials were taken and crushed separately to pass through a 45-mesh sieve for later use; (2) the raw material powder was taken according to the percentage of the composition components and mixed to obtain a mixture, and the mixture was added in deionized water with a material-to-liquid ratio of 1:10 (g/mL), and soaked for 30 min; (3) the material in step (2) was extracted at 70° C. for 3 h, filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a complex polysaccharide.

### Example 5

A complex polysaccharide was prepared by mixing the following raw materials in mass percentages: 40% of Agaricus Blazei Murill, 20% of Laminaria japonica, 5% of Glycyrrhiza glabra L., 15% of polygonatum and 20% of honeysuckle.

The complex polysaccharide was prepared by the following process: (1) the above raw materials were taken and crushed separately to pass through a 45-mesh sieve for later use; (2) the raw material powder was taken according to the percentage of the composition components and mixed to obtain a mixture, and a mixture was added in deionized water with a material-to-liquid ratio of 1:10 (g/mL), and soaked for 30 min; (3) the material in step (2) was extracted at 70° C. for 3 h, filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a complex polysaccharide.

### Comparative Example 1

A Glycyrrhiza glabra L. polysaccharide was prepared from (Glycyrrhiza glabra L. through the following process: (1) the raw material of Glycyrrhiza glabra L. was taken and crushed to pass through a 45-mesh sieve for later use; (2) the raw material powder was taken and added in deionized water with a material-to-liquid ratio of 1:15 (g/mL), to be soaked for 30 min; (3) the material in step (2) was extracted at 80° C. for 3 h, filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a polysaccharide component of a Glycyrrhiza glabra L. extract.

### Comparative Example 2

A polysaccharide was prepared from Agaricus Blazei Murill through the following process: (1) the raw material of Agaricus Blazei Murill was taken and crushed to pass through a 45-mesh sieve for later use; (2) the raw material powder was taken and added in deionized water with a material-to-liquid ratio of 1:15 (g/mL), to be soaked for 30 min; (3) the material in step (2) was extracted at 80° C. for 3 h. filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a polysaccharide component of an Agaricus Blazei Murill extract.

### Comparative Example 3

A polysaccharide was prepared from honeysuckle through the following process: (1) the raw material of honeysuckle was taken and crushed to pass through a 45-mesh sieve for later use; (2) the raw material powder was taken and added in deionized water with a material-to-liquid ratio of 1:15 (g/mL), to be soaked for 30 min; (3) the material in step (2) was extracted at 80° C. for 3 h, filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a polysaccharide component of a honeysuckle extract.

### Comparative Example 4

A polysaccharide was prepared from polygonatum through the following process: (1) the raw material of polygonatum was taken and crushed to pass through a 45-mesh sieve for later use; (2) the raw material powder was taken and added in deionized water with a material-to-liquid ratio of 1:15 (g/mL), to be soaked for 30 min; (3) the material in step (2) was extracted at 80° C. for 3 h, filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a polysaccharide component of a polygonatum extract.

### Comparative Example 5

A polysaccharide was prepared from Rhodophyta through the following process: (1) the raw material of Rhodophyta was taken and crushed to pass through a 45-mesh sieve for later use; (2) the raw material powder was taken and added in deionized water with a material-to-liquid ratio of 1:15 (g/mL), to be soaked for 30 min; (3) the material in step (2) was extracted at 80° C. for 3 h, filtered by a 300-mesh filter cloth, and a filtrate was collected; and (4) the filtrate in step (3) was concentrated to a viscous state at 70° C., 4 times of absolute ethanol was added according to the volume of the viscous liquid, alcohol precipitation and standing-still treatment were performed for 12 h, and a polysaccharide precipitate was collected by filtration, to obtain a polysaccharide component of a Rhodophyta extract.

### Example 6: Determination for Molecular Weight

10 mL of the polysaccharide extract sample prepared in Example 1 was precisely measured and placed in a 50 mL centrifuge tube, 40 mL of 60% ethanol was added to the centrifuge tube to be mixed well to obtain a mixture, and the mixture was stood still in a refrigerator at 4° C. for more than 2 h, and centrifuged at 4,000 r/min for 5 min, then supernatant was discarded, a polysaccharide extract was taken out to be evenly placed in a watch glass, and dried in a 60° C. drying oven for 4 h to 6 h until the sample was completely dried.

10 mg of the dried sample was precisely weighed and placed in a 1.5 mL centrifuge tube, a 2 mL of 0.1 mol/L sodium nitrate solution was added to be mixed well, the insoluble sample was placed in 60° C. hot water for accelerated dissolution, and after complete dissolution, centrifugation was performed for 10 min at 12,000 r/min, to obtain a sample to be tested.

Appropriate amounts of dextran with molecular weights of 1,000, 5,000, 12,000, 80,000, 150,000, 270,000, 670,000 and 2,000,000 were taken and weighed accurately to be prepared into a 4-5 mg/mL mixed standard solution. A TSKgel G4000PWxI chromatographic column (7.8 mm I.D.×30 cm, 10 μm) was adopted and a 0.1 mol/L sodium nitrate buffer solution served as a flowing phase to perform isocratic elution at 35° C. with a flow velocity of 0.5 mL/min.

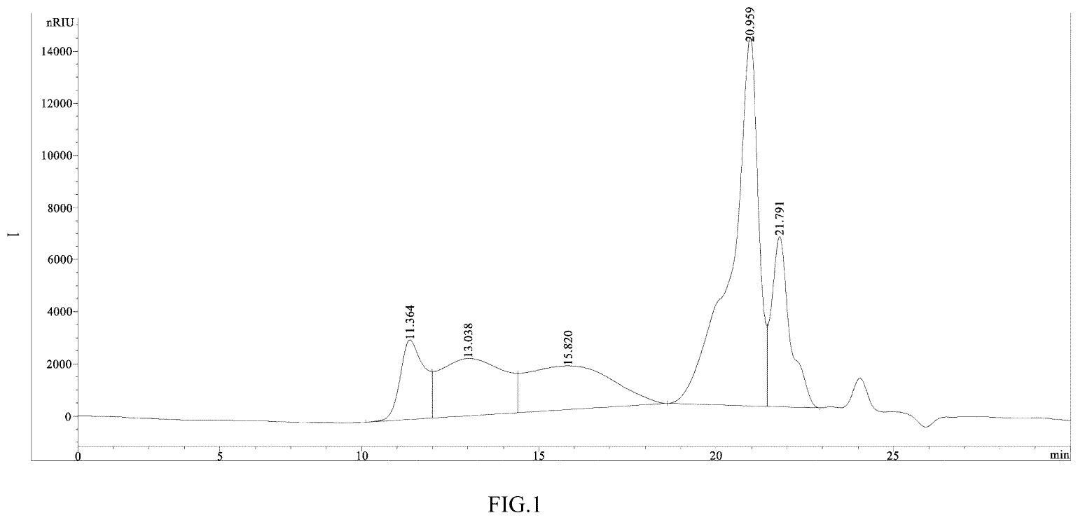

The results were shown in FIG. 1 and Table 1.

TABLE 1
Molecular weight distribution of the complex
polysaccharide prepared in Example 1

| | Weight average | Area |
|---|---|---|
| Retention time | molecular weight | percentage % |
| 11.3652 | 2,432,826 | 8.52252 |
| 13.0392 | 682,997 | 16.407 |
| 15.8202 | 80,286 | 16.8219 |
| 20.961 | 1,527 | 44.3044 |
| 21.7926 | 510 | 13.9441 |

It can be seen from FIG. 1 and Table 1 that the molecular weight distribution of the complex polysaccharide obtained in Example 1 ranged from 0.5 to 2432 KDa, and the weight average molecular weight was 321 KDa.

### Example 7: Determination for Monosaccharide Composition

10 mL of the polysaccharide extract sample prepared in Example 1 was precisely measured and placed in a 50 mL centrifuge tube, 40 mL of 60% ethanol was added to be mixed well to obtain a mixture, the mixture was stood still in a refrigerator at 4° C. for more than 2 h and centrifuged at 4,000 r/min for 5 min, then supernatant was discarded, a polysaccharide extract was taken out to be evenly placed in a watch glass, dried in a 60° C. drying oven for 4 h to 6 h until the sample was completely dried. 0.5 g of the dried sample was weighed precisely and placed in a 50 mL hydrolysis tube, a 5 mL of 4 mol/L trifluoroacetic acid solution was added, mixed well, and placed in an oven for hydrolysis at 110° C. for 1.5 h. After taking out and cooling the sample, a 4 mol/L sodium hydroxide solution was adopted to adjust the pH value to pH 7.0, the solution was transferred to a 50 mL volumetric flask, then diluted to volume with deionized water, and stored at −20° C. for later use. Appropriate amounts of mannose, lyxose, rhamnose, galacturonic acid, glucose, galactose, xylose, arabinose, fucose, fructose, and glucuronic acid reference substances were taken and weighed precisely to be prepared into a 500 mg/L mix9 mixed standard solution (calculated as a glucose concentration), then the solution was diluted into a series of standard curve solutions. 400 μL of sample and mixed standard solution were pipetted into a 5 mL test tube with stopper for derivatization. 400 μL of 0.5 mol/L PMP-methanol solution was added into the test tube, and then 400 L of 0.3 mol/L sodium hydroxide solution was added to be mixed well, and a reaction was performed in a 70° C. water bath for 100 min. After the end of the reaction, 500 μL of 0.3 mol/L hydrochloric acid solution was separately added to adjust the pH to neutral, shaken well and cooled to a room temperature, 1 mL of chloroform was added, with even vortex, centrifuged at 900 rpm for 5 min, a trichloride methane layer was discarded, supernatant was collected, the above operations were repeated for 5 times, and finally the supernatant was taken to pass through a 0.45 μm water-based membrane, and a filtrate was stored at −20° C. until sample injection.

Agilent C18 (4.6 mm×250 mm, 5 μm) was used for analysis, the detection wavelength was 250 nm, and the flow velocity was 1.0 mL/min, a 0.05 mol/L phosphate buffer solution served as a flowing phase A and acetonitrile served as a flowing phase B in an elution process, and gradient elution had the ratio as follows (flowing phase A:flowing phase B):0 min: 82%: 18%, 27 min: 20%: 80%, 43 min: 82%: 18%.

The results were shown in Table 2.

TABLE 2
Monosaccharide composition of the complex polysaccharide prepared in Example 1

| | | Galacturonic | | | | | |
|---|---|---|---|---|---|---|---|
| Mannose | Rhamnose | acid | Glucose | Galactose | Xylose | Arabinose | Fucose |
| 0.98% | 1.36% | 0.39% | 80.93% | 13.14% | 0.80% | 1.99% | 0.41% |

The results showed that the monosaccharide composition included mannose, rhamnose, galacturonic acid, glucose, galactose, xylose, arabinose, and fucose.

### Example 8: Characterization of Scanning Electron Microscope

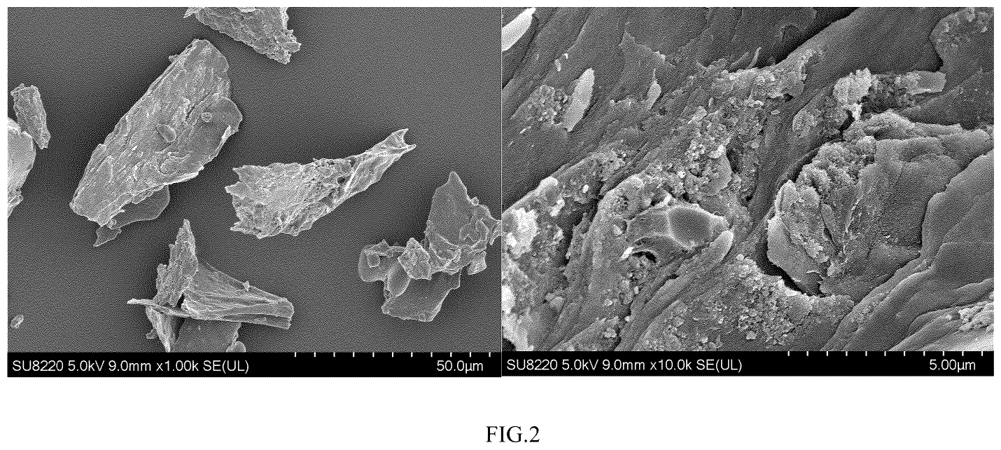

A trace amount of the complex polysaccharide sample was taken and stuck on a processing piece with a conductive tape. After spraying with gold, conductive analysis was performed, and the microscopic morphology of each polysaccharide was observed using an electron microscope. FIG. 2 showed an SEM scan of a complex polysaccharide of Example 1. The complex polysaccharide was in a mixed form of crystalline and amorphous, and had a lamellar structure, indicating that the complex polysaccharide prepared in Example 1 had the characteristics of multiple polysaccharide raw materials; and the complex polysaccharide contained a polysaccharide combination effective in regulating the immune system. Compared with the immunomodulating effect of a single polysaccharide, the complex polysaccharide had a more significant effect in regulating the Th1/Th2 immune balance.

### Example 9: Congo Red Test

A 5 mol/L NaOH solution was added to a mixture of 2 mL of the complex polysaccharide (1 mg/mL) and 2 mL of Congo red (80 μmol/L) in Example 1, so that the final concentrations of NaOH were 0 mol/L, 0.1 mol/L, 0.2 mol/L, 0.3 mol/L, 0.4 mol/L and 0.5 mol/L, and a UV spectrophotometer was used to scan the wavelength of 400 nm to 600 nm at each NaOH concentration, and the maximum absorption wavelength of the mixed solution at each NaOH concentration was recorded. Plotting was performed with distilled water as the blank control, the NaOH concentration as the abscissa, and the maximum absorption wavelength as the ordinate.

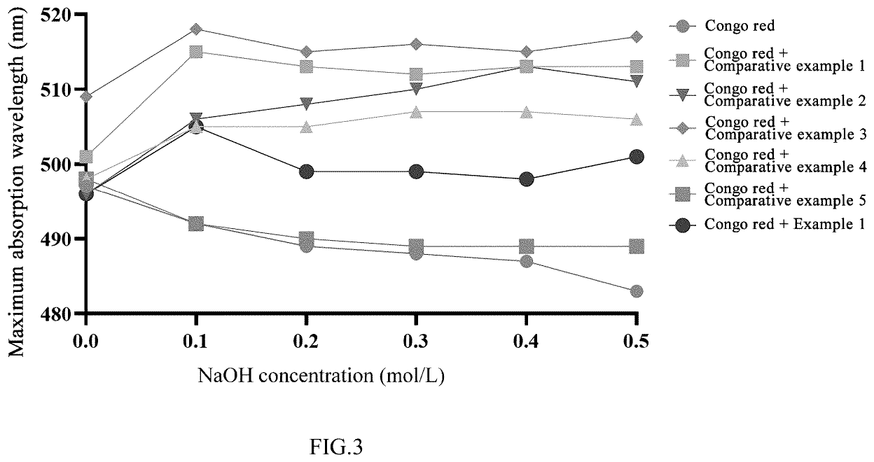

The tertiary-helix structure of polysaccharides was tightly closed to immunomodulatory activity. The Congo red test showed that compared with the single plant polysaccharides of Comparative examples 1-5, the tendency of the complex polysaccharides to first rise and then fall was more obvious, indicating that the complex polysaccharide had a more obvious tertiary-helix conformation, and better immunomodulatory activity (FIG. 3).

Test result 1: Inhibition of Immune Stress Induced by Ovalbumin

The efficacy verification test was conducted on the complex polysaccharide solid powder (hereinafter referred to as the complex polysaccharide) prepared according to the raw material ratio and process provided in Example 1. As a comparative experiment for synergy, single-component extraction of polysaccharides in Comparative examples 1-5 was set for comparison.

Test method: reference methods (YANG Ling, LIU Jie, L I Jiangping, L I He; Effect of ephedrine mediated TSLP/OX40L pathway in regulating Th2 type immune response in rats with allergic rhinitis [J]. Chinese Journal of Immunology, 2022, 38 (03): 319-323): ovalbumin (OVA) was used to create a model, and SD rats were used as model animals. 54 SD rats were randomly divided into 9 groups, 6 rats/group, which were respectively the blank group, model group, positive group (loratadine), Example 1 group, and single polysaccharide group (Comparative examples 1-5, five groups in total). Except for the blank group, the other test groups were intraperitoneally injected with OVA solutions in respective on days 1, 3, 5, 7, 9, 11, and 13 (0.3 mg ovalbumin (OVA), 30 mg AI (OH): mixed in 1 mL normal saline), to intensify sensitization. Beginning on the day 15, SD rats were subjected to nasal stimulation with OVA. Except for the blank group, 50 μL OVA (0.1 mg/μL) was dripped into the nostrils of each group of SD rat modeling groups for 7 days of continuous stimulation. On day 21 (after the last nasal instillation), the degree of symptoms of sneezing, scratching the nose, and running nose were counted. The scoring standards were calculated according to Table 3 for behavioral index results. The samples were collected after 24 h of stimulation on day 21. The levels of IL-13, IL-31, IgE, and HIS (histamine) in rat serum were detected.

TABLE 3
Scoring criteria for nasal symptoms in rats with allergic rhinitis

| | Mild | Moderate | Severe |
|---|---|---|---|
| Symptom | (1 point) | (2 points) | (3 points) |
| Nose scratching | 1-3 | 4-10 | >10 |
| frequency/30 minutes | | | |
| Number of sneezing/ | 1-4 | 5-10 | >10 |
| 30 minutes | | | |
| Running nose/minute | Flow to the | Drainage from | Tears streaming |
| | anterior | anterior nares | down the face |
| | nostril | | |

Intragastric administration was conducted for animal test for 21 days, and starting from the time of modeling, the specific settings were as follows: (1) the blank control group and model group were given 0.9% NaCl normal saline; (2) Example 1 group, complex polysaccharide (dosage: 200 mg/kg/d, solvent: 0.9% NaCl normal saline); (3) positive group, loratadine (dosage: 2 mg/kg/d, solvent: 0.9% NaCl normal saline): (4) single polysaccharide group (Comparative examples 1-5, five groups in total, dosage: 200 mg/kg/d, solvent: 0.9% NaCl normal saline), which were extracted and prepared in respective according to the process of Comparative examples 1-5.

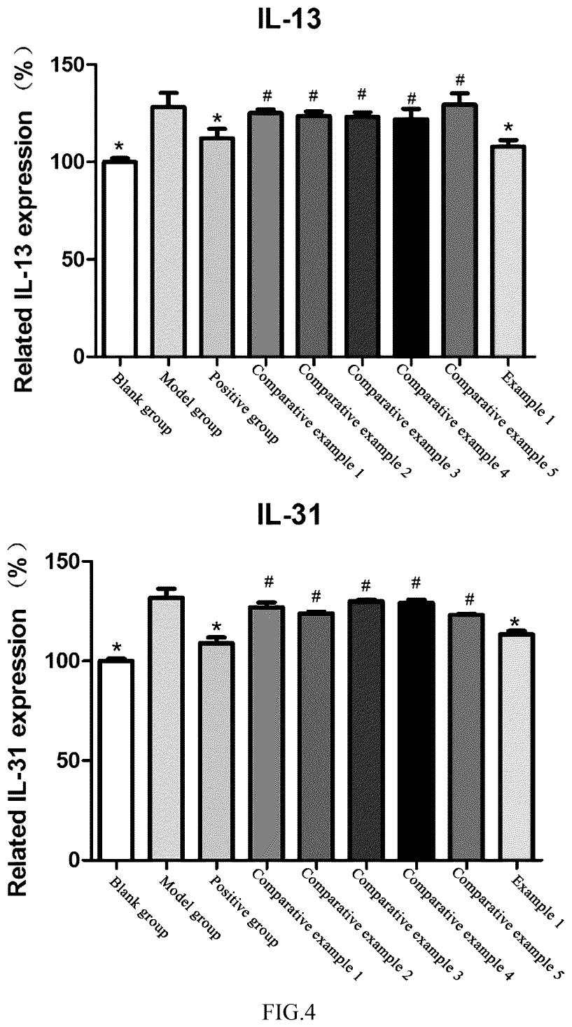

In the OVA model test, by monitoring the IL-13 and IL-31 indexes, it was found that IL-13 and IL-31 were respectively up-regulated to a certain extent after modeling, but after administration of polysaccharides in Comparative examples 1-5 and Example 1, both indexes declined, with the complex polysaccharide of Example 1 declining most obviously (FIG. 4 and Table 4).

TABLE 4
Expression levels of IL-13, IL-31, IgE and HIS in mouse serum

| Group | IL-13 (pg/mL) | IL-31(pg/mL) | IgE (μg/mL) | HIS (ng/mL) |
|---|---|---|---|---|
| Blank group | 28.76 ± 0.54* | 44.64 ± 1.26* | 1.98 ± 0.12* | 14.15 ± 0.26* |
| Model group | 36.87 ± 2.07 | 58.85 ± 4.90 | 3.16 ± 0.52 | 17.11 ± 0.49 |
| Positive group | 32.21 ± 1.47* | 48.65 ± 3.14* | 2.49 ± 0.15 | 14.60 ± 0.45* |
| Comparative | 36.00 ± 0.56^{#} | 56.73 ± 2.54^{#} | 2.85 ± 0.22 | 15.65 ± 0.40*^{#} |
| example 1 | | | | |
| Comparative | 35.58 ± 0.63^{#} | 55.35 ± 0.82^{#} | 2.82 ± 0.53 | 15.20 ± 0.16* |
| example 2 | | | | |
| Comparative | 35.42 ± 0.70^{#} | 58.03 ± 0.93^{#} | 3.26 ± 0.15^{#} | 16.84 ± 0.45^{#} |
| example 3 | | | | |
| Comparative | 35.09 ± 1.53^{#} | 57.73 ± 1.65^{#} | 2.93 ± 0.60 | 15.35 ± 0.31*^{#} |
| example 4 | | | | |
| Comparative | 37.22 ± 1.67^{#} | 54.98 ± 0.71^{#} | 2.64 ± 0.55 | 14.99 ± 1.22* |
| example 5 | | | | |
| Example 1 | 30.99 ± 0.98* | 50.56 ± 2.02* | 2.27 ± 0.19* | 14.26 ± 0.67* |

Note:
compared with the model group,
*P < 0.05; and compared with the Example 1 group,
^{#}p < 0.05.

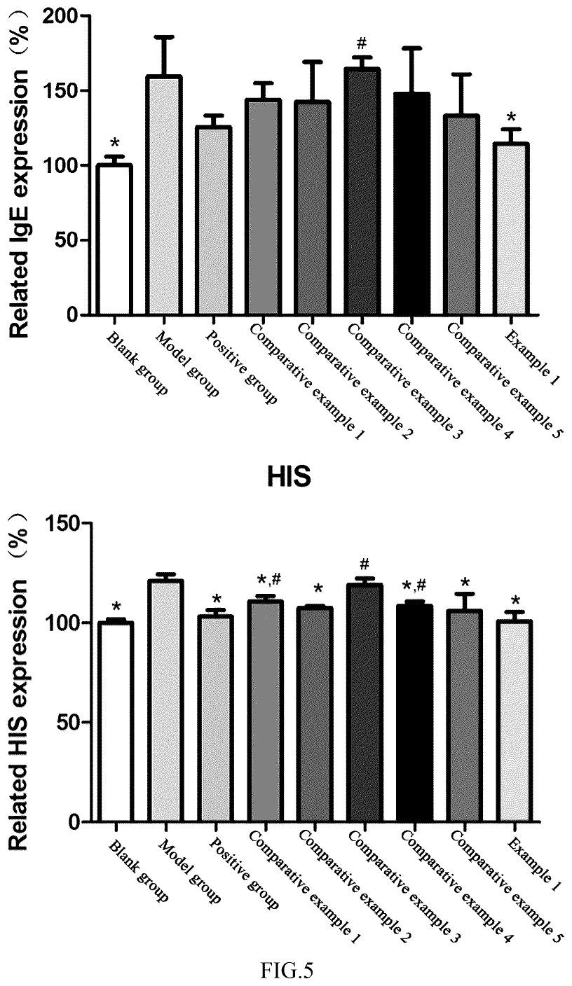

FIG. 5 showed relative expression levels of IgE and HIS in the serum of each group. Combined with Table 4 and FIG. 5, it can be seen that the Example 1 group could effectively reduce the expression of IgE and HIS factors in the serum of rats in a state of sexual immune stress, and had the role of simultaneously regulating multiple immune factors to achieve the synergistic purpose, and the effect was close to that of a positive drug. The effect of the complex polysaccharide of Example 1 was better than that of any group of Comparative examples 1-5.

Table 5 showed the behavioral index results of each test group; it can be seen from Table 5 that in the OVA model animal test, compared with the blank control group, the symptoms of sneezing, scratching the nose and running nose increased in the model group. Compared with the model group, these symptoms of rats in the Example 1 group were alleviated, and the effect was better than that of any one of Comparative examples 1-5 and the positive group.

TABLE 5
Behavioral index results of each test group (mean value)

| | Nose | | Running | |
|---|---|---|---|---|
| | scratching | Sneezing | nose | Aggregate |
| Test group | score | score | score | score |
| Blank group | 0.17 | 0.17 | 0 | 0.33 |
| Model group | 1.67 | 2.00 | 1.67 | 5.00 |
| Positive group | 0.50 | 0.50 | 0.33 | 1.33 |
| Comparative example 1 | 1.40 | 1.00 | 1.00 | 3.20 |
| Comparative example 2 | 0.67 | 0.50 | 0.33 | 1.50 |
| Comparative example 3 | 0.83 | 0.50 | 0.33 | 1.67 |
| Comparative example 4 | 0.67 | 0.50 | 0.33 | 1.50 |
| Comparative example 5 | 1.00 | 0.50 | 0.25 | 1.75 |
| Example 1 | 0.50 | 0.25 | 0.25 | 1.00 |

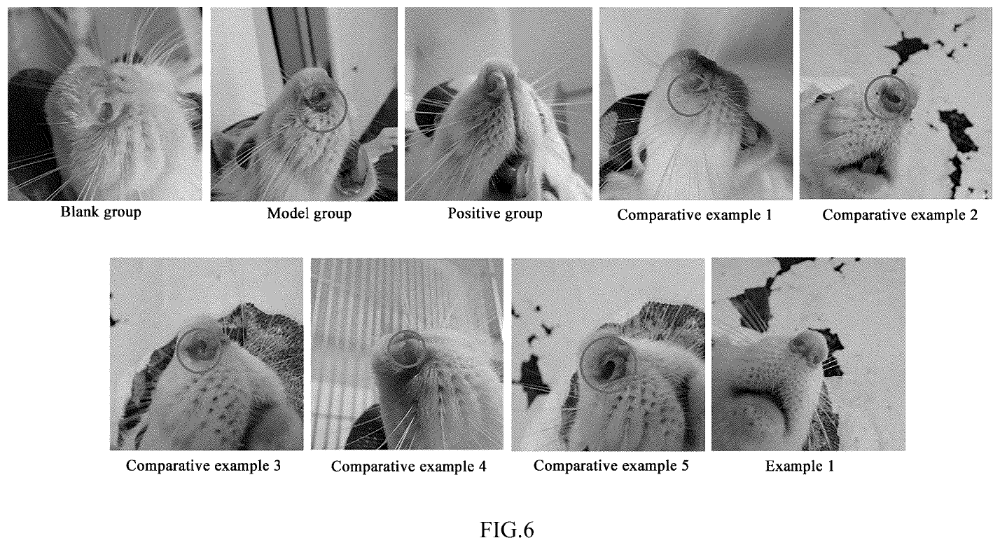

FIG. 6 showed a comparison of the anti-OVA-induced allergic rhinitis effects in this test. Examining the efficacy in terms of running nose, redness and swelling, it can be found that the complex polysaccharide was significantly better than any of Comparative examples 1-5 in terms of anti-OVA-induced allergic rhinitis, and had the obvious synergistic effects (the characteristics in the red circle).

Obviously, under the conditions of equal dosage, the inhibitory effect of the complex polysaccharide on IL-13, IL-31, IgE and HIS was better than any Comparative example. According to the calculation method of synergy index (Berenbaum index) (Berenbaum M C. The expected effect of a combination of agents: the general solution. Journal of Theoretical Biology, 1985, 114:413-431.):

$${\sum }_{i = 1}^{n} \frac{Xi}{Xie} ;$$

where Xi: the drug dosage of the i^{th} drug when used in combination; Xie: the dosage at which the i^{th} drug used alone can produce the same effect as used in combination; n: the number of drugs used in combination; and when the Berenbaum index was less than 1, it indicated a synergistic effect.

According to the mass percentage of the Chinese herbal medicine used in Example 1, the calculated Berenbaum index should be:

$$Berenbaum index = \frac{40 \% \times 200}{{X}_{1 e}} + \frac{10 \% \times 200}{{X}_{2 e}} + \frac{25 \% \times 200}{{X}_{3 e}} + \frac{20 \% \times 200}{{X}_{4 e}} + \frac{5 \% \times 200}{{X}_{5 e}} ;$$

where, “200” is the dosage of administration of the complex polysaccharide (mg/kg/d), and the other percentages represent the proportions of different single drugs in the complex polysaccharide. X₁ₑ, X₂ₑ, X₃ₑ, X₄ₑ, and X₅ₑ should be the dosage (mg/kg/d) capable of generating the same effect as the drug used in combination when Agaricus Blazei Murill, Rhodophyta, Glycyrrhiza glabra L., Polygonatum and honeysuckle were used alone. Obviously, when their values were “200”, the Berenbaum index was equal to 1; according to the solution of test 1, Example 1 and Comparative examples 1-5 were all tested at a dosage of 200 mg/kg/d. In Example 1, the regulatory effects of the model-induced IL-13, IL-31, IgE and HIS were better than those of administered alone. That is, the actual values of X₁ₑ, X₂ₑ, X₃ₑ, X₄ₑ, and X₅ₑ were all greater than 200, it can be estimated that the Berenbaum index of the complex polysaccharide in Example 1 under the OVA model was less than 1, that is, it had the synergistic effect. IL-13 and IL-31 were involved in the Th2 immune process. Therefore, the complex polysaccharide of Example 1 can down-regulate the expression of IgE and HIS by regulating the Th1/Th2 immune balance, thereby alleviating the development of OVA-induced allergic rhinitis symptoms, and having obvious synergistic effects.

The embodiments of the present disclosure is described in detail above in combination with the specific implementations, and however the present disclosure is not limited the above embodiments. Under the premise of not departing from the purpose of the present disclosure, various changes may also be made within the knowledge scope of those skilled in the art. In addition, the embodiments in the present disclosure and features in the embodiments may be combined with each other without conflict.

1. A polysaccharide composition, comprising a polysaccharide extract of an active pharmaceutical ingredient, wherein the active pharmaceutical ingredient comprises the following ingredients in mass percentage: 20%-40% of Agaricus Blazei Murill, 10%-30% of seaweed, 5%-25% of glycyrrhiza, 5%-25% of polygonatum and 5%-20% of honeysuckle.

2. The polysaccharide composition according to claim 1, wherein the active pharmaceutical ingredient comprises the following ingredients in mass percentage: 30%-40% of Agaricus Blazei Murill, 10%-20% of seaweed, 10%-25% of glycyrrhiza, 15%-25% of polygonatum and 5%-15% of honeysuckle.

3. The polysaccharide composition according to claim 1, wherein the seaweed is selected from at least one of Phaeophyta, Rhodophyta, Laminaria japonica, and Undaria pinnatifida Suringar.

4. The polysaccharide composition according to claim 1, wherein the glycyrrhiza is selected from at least one of Glycyrrhiza uralensis Fisch, Glycyrrhiza inflata Batal, and Glycyrrhiza glabra L.

5. A product for regulating the Th1/Th2 balance, comprising the polysaccharide composition according to claim 1.

6. A product for the prevention and/or treatment of Th1/Th2 imbalance-related diseases, comprising the polysaccharide composition according to claim 1, wherein the Th1/Th2 imbalance-related diseases comprise allergies and/or allergy-related diseases.

7. The product according to claim 6, wherein the allergy-related diseases comprise allergic rhinitis and allergic asthma.

8. The product according to claim 7, wherein the allergy comprises ovalbumin-induced allergy.

9. A product for enhancing immunity, comprising the polysaccharide composition according to claim 1.

10. The product according to claim 9, wherein the product comprises at least one of food, medicines and health care products.

11. A product, comprising the polysaccharide composition according to claim 1.

12. A preparation method of the polysaccharide composition according to claim 1, comprising the following steps: mixing raw materials after being crushed to obtain a mixture, adding water to soak the mixture for extraction, collecting a filtrate for concentration, alcohol precipitation, and collecting a precipitate.

13. The preparation method according to claim 12, wherein the extraction is performed for 2 h to 4 h at 70° C. to 90° C.

14. The preparation method according to claim 12, wherein the alcohol precipitation is that 3-5 volume times of absolute ethanol is added to stand still for 10 h to 14 h.
